(19) United States
(12) Patent Application Publication
(10) Pub. No.: US 2008/0171768 A1
(43) Pub. Date: Jul. 17, 2008

(54) Pharmaceutical Composition Containing An Anti Parasitic Agent And Active Ingredient Selected From Carveol, Thymol, Eugenol, Borneol, Carvacrol, Alpha-Ionone, Or Beta-Ionone

(75) Inventor: Adnane Remmal, Fes (MA)

Correspondence Address: SALIWANCHIK LLOYD & SALIWANCHIK;A PROFESSIONAL ASSOCIATION, PO BOX 142950, GAINESVILLE, FL, 32614-2950, US

(73) Assignee: ADVANCED SCIENTIFIC DEVELOPMENTS, Casablanca (MA)

(21) Appl. No.: 11/914,019

(22) PCT Filed: May 15, 2006
(86) PCT No.: PCT/IB06/01441

§ 371 Date: Jan. 22, 2008

(30) Foreign Application Priority Data
May 13, 2005 (IB) 2005/001314

(51) Int. Cl. A61K 31/4706; A61K 31/05; A61K 31/4709; A61P 33/00; A61K 31/35; A61K 31/357

(52) U.S. Cl. 514/313; 514/731; 514/450; 514/314; 514/455

(57) ABSTRACT

The present invention relates to a pharmaceutical composition comprising at least one first active therapeutic substance selected among carveol, thymol, eugenol, borneol, carvacrol, alpha-ionone, beta-ionone, as well as isomers, derivatives and mixtures thereof, and comprising at least one second active therapeutic substance that is an antiparasitic agent, in particular, an antimalarial agent. The invention is for use in the field of pharmaceutics.

The invention relates to a pharmaceutical composition comprising two therapeutically active substances one of which exerts a potentiating action on the other, and to the use of said composition.

It is known that the efficacy of therapeutic agents depends on the doses used which, in the case of partial resistance, necessitates increasing the doses of the therapeutic agents in order to attain the desired efficacy. This dose increase leads to problems with adverse effects and acute or chronic toxicity, which may considerably complicate the condition of the treated patients.

Said partial resistance may turn into complete resistance. In this case, increasing the dose no longer has any beneficial therapeutic effect; only the toxic effects are observed. The treatment in such a case consists in changing the therapeutic agent.

This chain of events can repeat itself and lead to the most serious situation: complete resistance to multiple therapeutic agents (multidrug resistance).

For instance, in particular, immunosuppressed patients become increasingly difficult to treat and their life expectancy is correspondingly shortened. Moreover, their quality of life is substantially affected by the administration of high doses of therapeutic agents.

The invention is directed at alleviating these problems by proposing to combine at least two therapeutically active substances, one of which potentiates the activity of the other, which not only makes it possible to lower the doses of each therapeutically active substance but also to treat patients afflicted with infections caused by resistant microorganisms.

In this regard, the invention provides a pharmaceutical composition characterized in that it comprises:
at least one first therapeutically active substance selected in the group consisting of carveol, thymol, eugenol, borneol, carvacrol, alpha-ionone, beta-ionone, and the isomers, derivatives and mixtures thereof,

and,
at least one second therapeutically active substance which is an antiparasitic agent.

The first therapeutic substance can be obtained by chemical synthesis or from a plant source.

Preferably, the antiparasitic agent in the inventive composition is selected in the group consisting of the antihelminthics, the protozoacides, and the mixtures thereof.

More preferably, the antiparasitic agent is selected in the group consisting of salinomycin, niclosamide, praziquantel or isoquinolein, albendazole, fubendazole, mebendazole, tiabendazole, triclabendazole, bithionol, diethylcarbamazine, ivermectin, levamisole, metrifonate, niclofan, oxamniquine, piperazine, pyrantel, pyrvinium, metronidazole, nimorazole, ornidazole, secnidazole, timidazole, meglumine antimoniate, pentamidine isethionate, sodium stibogluconate, benznidazole, difluoromethylornithine (DFMO), melarsoprol, nifurtimox, sodium suramin, amodiaquine, artemisine, artesunate and derivatives thereof, chloroquine, doxycycline, halofantrine, mefloquine, primaquine, proguanil, pyronaridine, quinine, atovaquone, azithromycin, clarithromycin, clindamycin, cotrimoxazole, dapsone, dehydroemetine, paromomycin, pyrimethamine, spiramycin, sulfadiazine, tenonitrozole, tiliquinol trimethoprim, trimethrexate, and the derivatives and mixtures thereof.

A more particularly preferred antiparasitic composition is a composition in which the first therapeutically active substance is thymol and the antiparasitic agent is salinomycin.

Another more particularly preferred antiparasitic composition is an antiparasitic composition in which said first therapeutically active substance is carvacrol and the antiparasitic agent is salinomycin.

Yet another more particularly preferred composition is an antimalarial composition in which said first therapeutically active substance is selected from carvacrol and eugenol, and the antiparasitic agent is an antimalarial selected from mefloquine, chloroquine, and artesunate, and the mixtures thereof.

The invention also proposes a kit characterized in that it comprises at least one first container containing a first therapeutically active substance selected in the group consisting of carveol, thymol, eugenol, borneol, carvacrol, alpha-ionone, beta-ionone, and the isomers and derivatives and mixtures thereof, and at least one second therapeutically active substance which is an antiparasitic agent.

The invention further proposes a method for treating an infection due to a parasite characterized in that one administers, simultaneously or sequentially, to a patient having a parasitic infection, at least one first therapeutically active substance selected in the group consisting of carveol, thymol, eugenol, borneol, carvacrol, alpha-ionone, beta-ionone, and the isomers and derivatives and mixtures thereof, and at least one second therapeutically active substance which is an antiparasitic agent.

Preferably, in said method, one simultaneously or sequentially administers to a patient having a parasitic infection between 10 and 100 mg/kg of body weight/day of said first therapeutically active substance, and between 20 and 100 mg/kg of body weight/day of second therapeutically active substance which is an antiparasitic agent.

Preferably, in this method, said first therapeutically active substance is selected in the group consisting of carvacrol, eugenol and thymol and said second therapeutically active substance is salinomycin.

The invention also proposes a method for treating malaria characterized in that one simultaneously or sequentially administers to a patient with malaria at least one first therapeutically active substance selected from eugenol or carvacrol, and at least one second therapeutically active substance which is an antimalarial selected in the group consisting of mefloquine, artesunate, chloroquine and the mixtures thereof.

The invention will be better understood and other aims and advantages thereof will appear more clearly in the explanatory description which follows.

The pharmaceutical composition according to the invention comprises as first therapeutically active substance thymol, eugenol, carvacrol, borneol, carveol, alpha-ionone, beta-ionone, and the derivatives and isomers as well as mixtures thereof.

Said substances must be pure.

Said compounds themselves have well-known antimicrobial properties.

Thymol, eugenol, carvacrol, borneol and carveol, alpha-ionone and beta-ionone are found in various proportions in different aromatic plant extracts, that is to say, they can be purified from such plants. However, they can quite simply be obtained by chemical synthesis.

As a matter of fact, the inventors have now discovered that said compounds have a potentiating effect on many therapeutically active substances including known antiparasitic agents which are already used as medicaments specific in this field.

The second therapeutically active substance comprised in the pharmaceutical composition of the invention is therefore an antiparasitic agent which is already known as such and already used as medicament specific in this field, and whose activity is potentiated.

Any other future antiparasitic agents can also be used

Examples of known antiparasitic agents already used as medicaments specific in this field which can be used in the pharmaceutical composition of the invention, and whose effect will be potentiated by the first pure therapeutically active substance, belong to two families: the antihelminthics and the protozoacides.

The antihelminthics family comprises the cestocides such as niclosamide and praziquantel, the benzimidazoles such as albendazole, flubendazole, mebendazole, tiabendazole, triclabendazole, and other antihelminthics, such as bithionol, diethylcarbamazine, ivermectin, levamisole, metrifonate, niclofan, oxamniquine, piperazine, pyrantel, pyrvinium.

The protozoacides family comprises the 5-nitroimidazole series, metronidazole, nimorazole, ornidazole, secnidazole, timidazole, the leishmanicides, such as meglumine antimoniate, pentamidine isethionate, sodium stibogluconate, the trypanosomicides, such as benznidazole, difluoromethylornithine (DMFO), melarsoprol, nifurtimox, sodium suramin, the antimalarials, such as amodiaquine, artemisine, chloroquine, doxycycline, halofantrine, mefloquine, primaquine, proguanil, pyronaridine, quinine, artesunate. Other protozoocidal agents are salinomycin, atovaquone, azithromycin, clarithromycin, clindamycin, cotrimoxazole, dapsone, dehydroemetine, paromomycin, pyrimethamine, spiramycin, sulfadiazine, tenonitrozole, tiliquinol trimethoprim, trimethrexate.

Said compounds can be used alone, or in combination with each other. The derivatives thereof, if they have antiparasitic activity, can also be used.

Particularly preferred are salinomycin, mefloquine, chloroquine and artesunate used in combination more particularly with carvacrol, eugenol and/or thymol.

Of course, the pharmaceutical composition according to the invention is not restricted to the use of only those antiparasitic agents mentioned above.

In fact, considering the potentiating effect exerted by the first therapeutically active substance defined in the invention, other known or future antiparasitic agents can also be successfully used.

The pharmaceutical composition according to the invention can be formulated so as to be suitable for a simultaneous or sequential administration of said at least first and second therapeutically active substances.

The pharmaceutical form of the pharmaceutical composition of the invention shall be adapted to its use.

For example, it can be used in the form of a solution, suspension, tablet or other.

The compositions for parenteral administration are generally pharmaceutically acceptable sterile solutions or suspensions which can optionally be prepared immediately before use.

For the preparation of nonaqueous solutions or suspensions, it is possible to use natural vegetable oils like olive oil, sesame oil or paraffin oil or the injectable organic esters such as ethyl oleate. The sterile aqueous solutions can be composed of a solution of therapeutically active substances in water. The aqueous solutions are suitable for intravenous administration in so far as the pH is properly adjusted and/or they are made isotonic, for example by adding a sufficient amount of sodium chloride or glucose.

In fact, considering the chemical structure of antiparasitic agents, and secondly, considering the chemical structure of carveol, carvacrol, thymol, eugenol, borneol, alpha-ionone and beta-ionone, it is thought, without intending to be bound by this theory, that carveol, carvacrol, thymol, eugenol, borneol, alpha-ionone and beta-ionone and the isomers, derivatives and mixtures thereof, interact with the antiparasitic agents to form complexes having a structure which diffuses more easily into the body's physiological fluids and which diffuses more easily into the cytoplasm of target infected cells.

However, it has been shown that when the different components of the pharmaceutical composition of the invention are mixed in the presence of detergents such as Tween or Triton or solvents such as ethanol or DMSO (dimethyl sulfoxide), the active molecules of the first and second therapeutically active substance associate with the molecules of the detergents and solvents and do not form potentiating complexes.

Now it has been discovered that the potentiating complex forms when an aqueous agar suspension is used, as means of dispersion by viscosity.

Thus, the pharmaceutical composition of the invention will preferably be prepared without detergent and without solvent. For example, it will be prepared as an aqueous suspension made viscous by the addition of agar at a non-solidifying concentration, for example from 1 to 5 grams of agar per liter of suspension.

The pharmaceutical composition of the invention enables the treatment of local or systemic infections caused by resistant microorganisms using doses of each of said first and second therapeutically active substance which are lower than the doses required for treating the same infections due to susceptible microorganisms with one or the other of these same said first and second therapeutically active substances alone.

In fact, the composition of the invention enables the use of doses of said first therapeutically active substance, when it is combined with said second therapeutically active substance, which are approximately three times lower than the doses required when said first therapeutically active substance is used alone, and of doses of said second therapeutically active substance, when it is combined with said first therapeutically active substance, which are two to ten times lower than the doses required when said second therapeutically active substance is used alone.

The result is to offer a treatment which has the following advantages:
effective at very low doses against susceptible microorganisms,
effective against microorganisms resistant to a therapeutic agent,
effective against microorganisms resistant to several therapeutic agents,
control of recurrence phenomena,
control of phenomena of resistant microorganisms selection,
in all these cases, there is a reduction in the risks of toxicity and/or adverse effects, well known to the person of the art, thanks to the administration of very low doses.

In addition, the costs of producing the treatment are reduced due to the use of small quantities of active substances used.

The pharmaceutical compositions according to the invention can be in the form of liposomes or associated with supports such as cyclodextrins or polyethylene glycols.

The pharmaceutical compositions of the invention are a simple and efficient means to combat the problems related to microbial agents in general which comprise mainly resistance to therapeutic agents and toxicity of the latter resulting from the use of high doses.

In fact, carveol, thymol, eugenol, borneol, carvacrol, alpha-ionone, beta-ionone and the derivatives, mixtures and isomers thereof, are simple molecules which have never been described as having any toxicity whatsoever and their addition with its potentiating effect on the second therapeutically active substance enables the use of much lower doses of said second therapeutically active substance.

In a first variant, then, the method for treating patients having a parasitic infection consists in administering to said patients the dose, determined by the physician, of the pharmaceutical composition of the invention comprising suitable doses of at least one said first therapeutically active substance, combined with suitable doses of at least one said second therapeutically substance, that is, the suitable antiparasitic agent.

In a second variant, the method for treating patients having a parasitic infection consists in sequentially administering to said patients the dose determined by the physician of at least one said first therapeutically active substance, followed by the suitable dose of at least one said second therapeutically active substance, that is, the suitable antiparasitic agent, or vice versa.

In this regard, the invention proposes a kit comprising at least one first container containing one of said first therapeutically active substances, and at least one second container containing one of said second therapeutically active substances.

Said kit enables health care personnel to prepare on demand either a mixture of suitable doses of the desired first therapeutic substance(s) and of the desired antiparasitic agent(s), for a simultaneous administration, or to sequentially and separately administer the suitable dose of at least one said first therapeutically active substance, followed by the suitable dose of at least one said second therapeutically active substance, that is, the suitable antiparasitic agent, or vice versa.

However, a mixture for simultaneous use shall be preferred in order to allow the potentiation complex to form and to act immediately after administration to the patient.

The invention will become clearer in the following examples describing different embodiments, which are given for purposes of illustration and not by way of limitation.

### EXAMPLE 1

### Treatment of Coccidiosis in Chickens Treated with Salinomycin Potentiated by Carvacrol or Thymol

These in vivo tests were designed to measure weight gain, consumption index, daily oocyst output and appearance of fecal matter in treated chickens.

The experiment was carried out on 200 chickens aged 22 days (average weight 620 g) afflicted with subclinical coccidiosis sampled from a group of 10,000 broiler chickens.

Subclinical coccidiosis was diagnosed on day 20. The mean oocyst output in the experimental animals was 50,000 oocysts/g (OPG).

Microscopic examination revealed the existence of two Eimeria species: Eimeria acervulina and Eimeria tenella.

The antiparasitic agent used is salinomycin, one of the most widely used antiparasitics for this type of infection. Two antiparasitic pharmaceutical compositions according to the invention were prepared by mixing salinomycin at different concentrations with carvacrol or thymol at a sub-inhibitory concentration of 75 mg/kg of feed.

Antiparasitic activity was also determined either with salinomycin alone, or with carvacrol alone or thymol alone. Antiparasitic activity was measured according to the conventional criteria for this type of infection: weight gain, consumption index, daily oocyst output and appearance of fecal matter. These criteria were measured on day 5 (immediately after stopping treatment) and on day 15 (10 days after stopping treatment).

The sample was subdivided into seven groups of 28 chickens each:

Group 1: Uninfected, untreated animals (healthy controls). Mean weight on day 22: 750 g.

Group 2: Infected, untreated animals (affection controls).

Group 3: Infected animals treated with salinomycin at a dose of 40 mg/kg of feed. Group 4: Infected animals treated with carvacrol 75 mg/kg of feed.

Group 5: Infected animals treated with thymol 75 mg/kg of feed.

Group 6: Infected animals treated with salinomycin 40 mg/kg of feed potentiated by carvacrol 75 mg/kg of feed.

Group 7: Infected animals treated with salinomycin 40 mg/kg of feed potentiated by thymol 75 mg/kg of feed.

Industrial (finishing) feed used does not contain any antiparastic agents.

The antiparasitic agents used were mixed with this feed during the five days of treatment. The animals had free access to the feed throughout the experiment.

Table 1 gives the results of the test to compare the action of the composition of the invention versus salinomycin alone, carvacrol alone and thymol alone.

TABLE 1

| | Weight gain (grams ± SD) | | Consumption index | | Oocyst output (OPG ± SD) | | Fecal appearance | |
|---|---|---|---|---|---|---|---|---|
| | Day 5 | Day 15 | Day 5 | Day 15 | Day 5 | Day 15 | Day 5 | Day 15 |
| Group 1 | 250 ± 24 | 1000 ± 70 | 1.62 | 1.82 | 1200 ± 158 | 1500 ± 115 | Normal | Normal |
| Uninfected, | | | | | | | | |
| untreated | | | | | | | | |
| controls | | | | | | | | |
| Group 2 | 168 ± 42 | 780 ± 97 | 2.3 | 3.1 | 92,000 ± 5500 | 194,000 ± 7800 | Diarrheal | Diarrheal |
| Infected, | | | | | | | | |
| untreated | | | | | | | | |
| controls | | | | | | | | |
| Group 3 | 182 ± 28 | 835 ± 44 | 2.2 | 2.9 | 110,000 ± 6700 | 164,000 ± 7200 | Diarrheal | Diarrheal |
| Salinomycin | | | | | | | | |
| alone (40 | | | | | | | | |
| mg/kg feed) | | | | | | | | |
| Group 4 | 176 ± 32 | 812 ± 51 | 2.4 | 3 | 105,000 ± 5900 | 147,000 ± 4300 | Diarrheal | Diarrheal |
| Carvacrol | | | | | | | | |
| alone (75 | | | | | | | | |
| mg/kg feed) | | | | | | | | |
| Group 5 | 180 ± 52 | 840 ± 45 | 2.45 | 3.3 | 98,000 ± 4200 | 136,000 ± 6500 | Diarrheal | Diarrheal |
| Thymol | | | | | | | | |
| alone (75 | | | | | | | | |
| mg/kg feed) | | | | | | | | |
| Group 6 | 230 ± 33 | 1120 ± 45 | 1.65 | 1.72 | 7240 ± 650 | 5400 ± 340 | Normal | Normal |
| Salinomycin | | | | | | | | |
| (SAL-P) 40 | | | | | | | | |
| mg/kg + | | | | | | | | |
| carvacrol 75 | | | | | | | | |
| mg/kg | | | | | | | | |
| Group 7 | 220 ± 41 | 1085 ± 56 | 1.70 | 1.78 | 9300 ± 570 | 6200 ± 450 | Normal | Normal |
| Salinomycin | | | | | | | | |
| (SAL-P) 40 | | | | | | | | |
| mg/kg + | | | | | | | | |
| thymol 75 | | | | | | | | |
| mg/kg | | | | | | | | |

Table 1 shows that the compositions of the invention had notable antiparasitic activity on these two Eimeria strains, as compared with salinomycin alone, with carvacrol alone or with thymol alone.

In fact, the potentiation of salinomycin by carvacrol and thymol gives surprising results in vivo regarding the considerable increase in the antiparasitic activity of salinomycin.

Indeed, all infected animals treated either with salinomycin alone, carvacrol alone or thymol alone are still diarrheal 15 days after stopping treatment, in addition to a smaller weight gain compared to uninfected and untreated controls.

On the other hand, when the animals were treated with the composition of the invention, their weight gain, consumption index and fecal appearance were identical to those seen in uninfected untreated controls.

### EXAMPLE 2

### Intrinsic Antimalarial Action of Eugenol and Carvacrol and Potentiation of Artesunate

In Vitro Test: Determination of IC₅₀ (Concentration Required for 50% Inhibition of Parasite Growth)

The experiment was carried out with four clones (named 3d7, HB3, Dd2 and 7G8) of the malarial agent Plasmodium falciparum, a parasitic disease also known as malaria. These clones are laboratory reference models commonly used to screen drugs for antimalarial activity.

Plasmodium falciparum was grown on human red blood cells. The test was carried out in 96-well plates in which infected red blood cells (synchronized with sorbitol) were contacted with the different treatments for 72 hours at 37° C. in an incubator with controlled atmosphere (5% CO₂, 1% O₂, 94% N₂). Growth of the pathogen was measured by quantifying fluorescent DNA in the presence of SybrGreen.

Artesunate, one of the most effective antiparasitic agents, is tested. An antiparasitic pharmaceutical composition according to the invention was prepared by mixing artesunate at different concentrations with carvacrol or eugenol at sub-inhibitory concentrations of 0.05 mM and 0.2 mM, respectively. These concentrations are two to six times lower (depending on the clone) than the IC₅₀ of carvacrol or eugenol alone. This pharmaceutical composition according to the invention was named artesunate P eugenol or artesunate P carvacrol, the letter P signifying potentiation by eugenol or carvacrol.

In each case, antiparasitic activity was determined either with artesunate alone, or with carvacrol alone, or with eugenol alone, or with the composition of the invention.

The IC₅₀ was determined with Harald Noedl's HN NonLin V1.051 Beta software based on the fluorescence values.

Tables 2 and 3 give the results of tests to determine the IC₅₀ values of the different compositions.

TABLE 2

| | | IC₅₀ composition | |
|---|---|---|---|
| | | of the invention | |
| Plasmodium | IC₅₀ | (artesunate-P | IC₅₀ |
| falciparum clones | artesunate alone | carvacrol) | carvacrol alone |
| in growth phase | nM | (nM artesunate) | nM |
| Clone 3d7 | 2.14 | 1.025 | 0.33 |
| Clone HB3 | 2.28 | 1.15 | 0.22 |
| Clone Dd2 | 2.66 | 1.25 | 0.16 |
| Clone 7G8 | 1.00 | 0.47 | 0.11 |

TABLE 3

| | | IC₅₀ composition | |
|---|---|---|---|
| | | of the invention | |
| Plasmodium | IC₅₀ | (artesunate-P | IC₅₀ |
| falciparum clones | artesunate alone | eugenol) | eugenol alone |
| in growth phase | nM | (nM artesunate) | nM |
| Clone 3d7 | 2.14 | 1.025 | 1.25 |
| Clone HB3 | 2.28 | 1.15 | 0.66 |
| Clone Dd2 | 2.66 | 1.40 | 0.66 |
| Clone 7G8 | 1.25 | 0.50 | 0.40 |

Tables 2 and 3 clearly indicate that both carvacrol and eugenol had a notable intrinsic antiparasitic activity at fairly low concentrations.

These tables also show that the composition of the invention had notable antimalarial activity on these clones with different susceptibilities, as compared with artesunate alone, with carvacrol alone or with eugenol alone at the subinhibitory concentrations used in this test.

In fact, it can be seen that by using a carvacrol concentration of 0.05 mM and a eugenol concentration of 0.2 mM, which are approximately two to six times lower than the IC₅₀ of carvacrol or eugenol alone, the artesunate concentration producing 50% inhibition was reduced almost by half.

It should be noted that in this example the antimalarial action of potentiated artesunate according to the invention was only two times superior to that of artesunate alone because the four clones were already susceptible to artesunate alone. In spite of this, the potentiation led to a doubling of the antimalarial activity.

### EXAMPLE 3

### Intrinsic Antimalarial Action of Eugenol and Potentiation of Chloroquine

In Vitro Test: Determination of IC₅₀ (Concentration Required for 50% Inhibition of Parasite Growth)

The experiment was carried out with the same Plasmodium falciparum clones as in example 2 (3d7, HB3, Dd2 and 7G8), the first two clones (3d7 and HB3) being susceptible and the other two (Dd2 and 7G8) being resistant to chloroquine.

Chloroquine, one of the most widely used antiparasitic agents, is tested. An antiparasitic pharmaceutical composition according to the invention was prepared by mixing chloroquine at different concentrations with eugenol at a sub-inhibitory concentration of 0.2 mM, which is two to six times lower (depending on the clone) than the IC₅₀ of eugenol alone. This pharmaceutical composition was named chloroquine P eugenol, the letter P signifying potentiation by eugenol.

In each case, antiparasitic activity was determined either with chloroquine alone, or with eugenol alone, or with the composition of the invention.

The IC₅₀ was determined as in example 2.

Table 4 gives the results of tests to determine the IC₅₀ values of the different compositions.

TABLE 4

| | | IC₅₀ composition | |
|---|---|---|---|
| | | of the invention | |
| Plasmodium | IC₅₀ | (chloroquine P | IC₅₀ |
| falciparum clones | chloroquine alone | eugenol) | eugenol alone |
| in growth phase | nM | (nM chloroquine) | nM |
| Clone 3d7 | 22.31 | 13.2 | 1.25 |
| Clone HB3 | 37.07 | 15.3 | 0.66 |
| Clone Dd2 | 493.84 | 12.5 | 0.66 |
| Clone 7G8 | 445.17 | 12.5 | 0.40 |

Table 4 clearly shows that eugenol had a notable intrinsic antiparasitic activity at a fairly low concentration.

Table 4 also shows that the composition of the invention had notable antimalarial activity on these clones with different susceptibilities, as compared with chloroquine alone or with eugenol alone.

In fact, it can be seen that by using 0.2 mM eugenol, which is approximately two to six times lower than the IC₅₀ of eugenol alone, the chloroquine concentration producing 50% inhibition was practically reduced by half for the two susceptible clones (3d7 and HB3) and by nearly 20-fold for the two resistant clones (Dd2 and 7G8).

### EXAMPLE 4

### Intrinsic Antimalarial Action of Eugenol and Potentiation of Mefloquine

In Vitro Test: Determination of IC₅₀ (Concentration Required for 50% Inhibition of Parasite Growth)

The experiment was carried out with the same Plasmodium falciparum clones as in examples 2 and 3 (3d7, HB3, Dd2 and 7G8), the first two clones (3d7 and HB3) being susceptible and the other two (Dd2 and 7G8) being resistant to mefloquine.

Mefloquine, one of the most widely used antiparasitic agents, is tested. An antiparasitic pharmaceutical composition according to the invention was prepared by mixing mefloquine at different concentrations with eugenol at a sub-inhibitory concentration of 0.2 mM, which is two to six times lower (depending on the clone) than the IC₅₀ of eugenol alone. This pharmaceutical composition according to the invention was named mefloquine P eugenol, the letter P signifying potentiation by eugenol.

In each case, antiparasitic activity was determined either with mefloquine alone, or with eugenol alone, or with the composition of the invention.

The IC₅₀ was determined as in examples 2 and 3.

Table 5 gives the results of tests to determine the IC₅₀ values of the different compositions.

TABLE 5

| | | IC₅₀ composition | |
|---|---|---|---|
| | | of the invention | |
| Plasmodium | IC₅₀ | (mefloquine P | IC₅₀ |
| falciparum clones | mefloquine alone | eugenol) | eugenol alone |
| in growth phase | nM | (nM mefloquine) | nM |
| Clone 3d7 | 8.83 | 2.79 | 1.25 |
| Clone HB3 | 8.96 | 3.48 | 0.66 |
| Clone Dd2 | 33.75 | 3.6 | 0.66 |
| Clone 7G8 | 15.32 | 2.67 | 0.40 |

Table 5 clearly shows that eugenol had a notable intrinsic antiparasitic activity at a fairly low concentration.

Table 5 also shows that the composition of the invention had notable antimalarial activity on these clones with different susceptibilities, as compared with mefloquine alone or with eugenol alone.

In fact, it can be seen that by using 0.2 mM eugenol, which is approximately two to six times lower than the IC₅₀ of eugenol alone, the mefloquine concentration producing 50% inhibition was reduced nearly three-fold for the two susceptible clones (3d7 and HB3) and 5 to 10-fold for the two resistant clones (Dd2 and 7G8).

### EXAMPLE 5

### Intrinsic Antimalarial Action of Eugenol and Potentiation of Artesunate

In vivo test:

Here, the intrinsic antimalarial activity of carvacrol as well as artesunate potentiated by carvacrol was studied in vivo in an animal model of cerebral malaria (neuromalaria). The reference model for this type of infection makes use of CBA/J mice infected with Plasmodium berghei ANKA, an agent that causes cerebral malaria.

The intraperitoneal route was used for infection and treatment of the mice.

In this model, the onset of the first clinical signs occurs by day 4 or 5 (D4 or D5) after infection. Animals presenting these signs die in the next two days (D6 and D7).

The test was carried out on five groups of 10 mice as follows:

Group 1: Uninfected, untreated controls

Group 2: Infected mice treated with carvacrol (60 mg/kg of weight) twice a day every 12 hours

Group 3: Infected mice treated with carvacrol (40 mg/kg of weight) twice a day every 12 hours

Group 4: Infected mice treated with artesunate (40 mg/kg of weight) once a day

Group 5: Infected mice treated with artesunate (40 mg/kg of weight) once a day and with carvacrol (40 mg/kg of weight) twice a day.

Treatment commenced five days after infection (D5) and continued for three days (D5, D6, D7).

Table 6 presents the results of this experiment.

TABLE 6

| | | Survival time | |
|---|---|---|---|
| | | of animals that | % survivor |
| | % mortality | died (days) | at D12 |
| Group 1 (controls) | 100 | 5.5 | N/A |
| Group 2 (carvacrol 120) | 20 | 7.5 | 80 |
| Group 3 (carvacrol 80) | 100 | 7.5 | N/A |
| Group 4 (artesunate 40) | 100 | 7 | N/A |
| Group 5 (artesunate 40 + | 0 | N/A | 100 |
| carvacrol 80) | | | |

*N/A: not applicable

Table 6 clearly shows that the administration of carvacrol alone at a dose of 60 mg/kg of animal weight twice a day can protect 80% of the treated animals, thus demonstrating a notable antimalarial activity of carvacrol alone at this dose (120 mg/kg of weight/24 h).

A treatment with carvacrol at a dose of 40 mg/kg twice a day does not afford any protection but slightly prolonged the survival time of treated animals, demonstrating partial therapeutic efficacy at the dose used (80 mg/kg of weight/24 h).

Artesunate alone (40 mg/kg of weight/24 h) does not provide any protection either but extends the survival time of infected animals, thereby demonstrating partial therapeutic efficacy.

The combination of artesunate (40 mg/kg) once a day and carvacrol (40 mg/kg) twice a day protected 100% of the animals beyond the incubation period for cerebral malaria, i.e., beyond D12.

These data clearly show that carvacrol alone exhibits notable antimalarial activity at the dose of 60 mg/kg twice a day.

They also clearly indicate that the composition of the invention exhibits notable antimalarial activity as compared with artesunate alone or with carvacrol alone.

Of course, the invention is in no way restricted to the embodiments described herein which are given solely for purposes of illustration and not by way of limitation.

On the contrary, the invention comprises all the technical equivalents of the means described herein as well as the combinations thereof where such are carried out in the spirit of the invention.

1-13. (canceled)

14. A pharmaceutical composition comprising:
at least one first therapeutically active substance selected from the group consisting of carveol, thymol, eugenol, borneol, carvacrol, alpha-ionone, beta-ionone, and isomers, derivatives or mixtures thereof, and
at least one second therapeutically active substance which is an antiparasitic agent selected from the group consisting of antihelminthics, protozoacides and derivatives or mixtures thereof.

15. The composition according to claim 14, wherein said second therapeutically active substance is selected from the group consisting of salinomycin, artesunate, mefloquine, chloroquine, and derivatives or mixtures thereof.

16. The composition according to claim 14, wherein said first therapeutically active substance is selected from the group consisting of thymol, eugenol and carvacrol, and isomers, derivatives or mixtures thereof and said second therapeutically active substance is selected in the group consisting of salinomycin, artesunate, mefloquine, chloroquine, and derivatives or mixtures thereof.

17. The composition according to claim 16, wherein said first therapeutically active substance is thymol or carvacrol and said second therapeutically active substance is salinomycin.

18. The composition according to claim 16, wherein said first therapeutically active substance is selected from carvacrol or eugenol and said second therapeutically active substance is selected from the group consisting of salinomycine, artesunate, mefloquine, chloroquine and mixtures thereof.

19. The composition according to claim 18, wherein said second therapeutically active substance is artesunate.

20. The composition according to claim 18, wherein said first therapeutically active substance is eugenol and said second therapeutically active substance is selected from the group consisting of artesunate, mefloquine, chloroquine and mixtures thereof.

21. The composition according to claim 17, wherein said first and second therapeutically active substances are suspended in an aqueous agar solution.

22. The composition according to claim 19, wherein said first and second therapeutically active substances are suspended in an aqueous agar solution.

23. The composition according to claim 20, wherein said first and second therapeutically active substances are suspended in an aqueous agar solution.

24. A kit comprising:
at least one first container containing a first therapeutically active substance selected from the group consisting of carveol, thymol, eugenol, borneol, carvacrol, alpha-ionone, beta-ionone, and isomers, derivatives or mixtures thereof; and
at least one second container containing a second therapeutically active substance which is an antiparasitic agent selected from the group consisting of salinomycin, artesunate, mefloquine, chloroquine, and derivatives or mixtures thereof.

25. The kit according to claim 24, wherein said first therapeutically active substance is selected from the group consisting of thymol, eugenol and carvacrol, and isomers, derivatives or mixtures thereof.

26. The kit according to claim 24, wherein said first therapeutically active substance is thymol or carvacrol and said second therapeutically active substance is salinomycin.

27. The kit according to claim 24, wherein said first therapeutically active substance is selected from carvacrol or eugenol and said second therapeutically active substance is selected from the group consisting of salinomycine, artesunate, mefloquine, chloroquine and mixtures thereof.

28. The kit according to claim 27, wherein said second therapeutically active substance is artesunate.

29. The kit according to claim 24, wherein said first therapeutically active substance is eugenol and said second therapeutically active substance is selected from the group consisting of artesunate, mefloquine, chloroquine and mixtures thereof.

30. A method for treating an infection caused by a parasite comprising simultaneously or sequentially administering to a patient having a parasitic infection:
at least one first therapeutically active substance selected from the group consisting of carveol, thymol, eugenol, borneol, carvacrol, alpha-ionone, beta-ionone and isomers, derivatives or mixtures thereof, and
at least one second therapeutically active substance which is an antiparasitic agent selected from the group consisting of salinomycin, artesunate, mefloquine, chloroquine, and derivatives or mixtures thereof.

31. The method according to claim 30, wherein said first therapeutically active substance is selected from the group consisting of thymol, eugenol and carvacrol, and isomers, derivatives or mixtures thereof.

32. The method according to claim 30, wherein said first therapeutically active substance is thymol or carvacrol and said second therapeutically active substance is salinomycin.

33. The method according to claim 30, wherein said first therapeutically active substance is selected from carvacrol or eugenol and said second therapeutically active substance is selected from the group consisting of salinomycine, artesunate, mefloquine, chloroquine and mixtures thereof.

34. The method according to claim 33, wherein said second therapeutically active substance is artesunate.

35. The method according to claim 33, wherein said first therapeutically active substance is eugenol and said second therapeutically active substance is selected from the group consisting of artesunate, mefloquine, chloroquine and mixtures thereof.

36. The method according to claim 30, wherein said method comprises simultaneously or sequentially administering to a patient having a parasitic infection:
between 10 and 100 mg/kg of body weight/day of said first therapeutically active substance; and
between 20 and 100 mg/kg of body weight/day of said second therapeutically active substance which is an antiparasitic agent.
